(19) United States
(12) Patent Application Publication
(10) Pub. No.: US 2026/0315018 A1
(43) Pub. Date: Oct. 15, 2026

(54) DETECTOR HOLDING DEVICE FOR AN X-RAY DEVICE AND X-RAY DEVICE

(71) Applicant: Siemens Healthineers AG, Forchheim (DE)

(72) Inventors: Andreas LIMMER, Fuerth (DE); Thomas DIPPL, Pressath (DE); Florian RUPPRECHT, Ingolstadt (DE); Susanne OEPPING, Bubenreuth (DE); Sandra ANTI, Fuerth (DE); Michael FUHRMANN, Herzogenaurach (DE); Sophia Katharina HEROLD, Maktzeuln (DE); Joerg HOFMANN, Forchheim (DE); Gerben TEN CATE, Hausen OT Wimmelbach (DE)

(73) Assignee: Siemens Healthineers AG, Forchheim (DE)

(21) Appl. No.: 19/643,204

(22) Filed: Apr. 9, 2026

(30) Foreign Application Priority Data
Apr. 10, 2025 (EP) 25169686.0

(51) Int. Cl. A61B 6/00

(52) U.S. Cl. CPC A61B 6/4452; A61B 6/4464

(57) ABSTRACT

A detector holding device for an X-ray device, the detector holding device comprising: an X-ray detector and a stand arrangement by which the X-ray detector is carried inside a housing of a detector module. The X-ray detector has a detector surface extending in a detector plane and is arranged behind a radiolucent wall of the housing. The X-ray detector is configured to move in the detector plane inside the housing in at least one direction via at least one selectable shift actuator.

### CROSS-REFERENCE TO RELATED APPLICATION(S)

The present application claims priority under 35 U.S.C. § 119 to European Patent Application No. 25169686.0, filed Apr. 10, 2025, the entire contents of which are incorporated herein by reference.

### FIELD

One or more example embodiments of the present invention relate to a detector holding device for an X-ray device comprising an X-ray detector and a stand arrangement by which the X-ray detector is carried inside a housing of a detector module, wherein the X-ray detector has a detector surface extending in a detector plane and arranged behind a radiolucent wall, in particular a panel, of the housing. One or more example embodiments of the present invention also relate to an X-ray device.

### BACKGROUND

Modern X-ray devices for radiography are designed to support different types of image acquisition techniques, for example both images acquired with a standing patient and images acquired with a lying or sitting patient. In order to achieve such flexibility, radiographic X-ray devices are typically provided with a plurality of components. Components of said type firstly comprise a tube assembly holding device for supporting an X-ray tube assembly, which holding device can be mounted on a ceiling of a room in which the X-ray device is arranged in order for example to allow the most efficient utilization of space possible. Detector holding devices for generating X-ray images of standing or seated patients are already known in the prior art in the form of grid wall units called “bucky wall stands” (BWS). These typically comprise a stand arrangement, for example consisting of a vertically extending column on which a height-adjustable detector module is arranged. In particular, the detector module is moved via a carriage mounted on a guide which extends over the height of the column. In this setup, the detector module typically comprises a wall which is permeable to X-rays and behind which the X-ray detector (image receptor), nowadays usually a digital X-ray detector, is arranged. The radiolucent wall is designed in the form of a panel and may therefore also be referred to as a panel. An antiscatter grid is also often provided between the X-ray detector and the radiolucent wall. The detector module may further comprise dose measuring chambers, in particular for automatic exposure control.

The lifting movement of the detector module on the column can in this case be achieved manually and/or automatically, for example via a motor or other suitable actuator. It is also known to arrange the detector module on the column so as to be tiltable, for example via a tilt unit, in order to enable the detector module and hence the X-ray detector to be tilted in a range, for example by +90° to −20°. When tilted by +90°, the radiolucent wall/panel is aligned horizontally, just like the detector surface, such that for example X-ray images of the arm or hand acquired with seated patients are possible. Foot and leg X-ray images are likewise possible. The other extreme position is used for specialized X-ray image acquisitions. Grab handles which a patient can hold on to can be arranged on the detector module. It has also already been proposed to use a bucky wall stand in order to produce X-ray images which cover a greater area than the detector surface of the X-ray detector and are composed of individual images stitched together by positioning the detector module together with the X-ray detector along the patient in the height direction for the different individual images. With such acquisition procedures or other applications in which a movement of the detector module takes place, a patient assist device is typically provided which supports the patient when assuming and maintaining the correct exposure position. Such a patient assist device may for example comprise steps and/or a chair, an additional wall placed in front of the moving detector module, and special grab handles. However, such a patient assist device forms an additional accessory which also makes the design of the X-ray device more complex.

In particular in tilted positions of the detector module, but also when additional aids such as the cited patient assist device are used, collisions can easily occur between the stand arrangement, in particular the column, and other components, in particular parts of the tube assembly holding device. This restricts the operating range and requires high levels of alertness on the part of the staff in order to avoid collisions. In the case of X-ray images taken with the detector module standing vertically, the patient must be precisely positioned in the transverse direction since the examination region often makes use of the whole of the detector surface.

But further problems also exist in relation to the currently known bucky wall stands. For instance, the height of the bucky wall stand, as well as the lifting movement of the bucky wall stand is limited, in particular due to the embodiment and size of the detector module. Since the stand arrangement is fixedly mounted on the floor of the room in which the X-ray device is installed, a repositioning of the patient is necessary in the case of examination regions offset transversely on the patient. The detector size is in this case and generally limited in the detector slide-in module.

There are also difficulties in the handling of known bucky wall stands. For example, cleaning such a detector holding device is difficult due to additional accessories and with known cladding elements. The antiscatter grid must be inserted and, if it is not required, removed manually. With a manually possible movement of the detector module on the stand arrangement, in particular the column, a counterbalance is necessary. A high amount of installation space is required in particular when an element known as a spacer box is provided in the horizontal direction between the stand arrangement, in particular the column, and the detector module in order for example to enable a patient support and positioning device to be suitably placed in relation to the detector module. No automatic workflow is possible when the provided degrees of freedom are actuated manually.

Also often provided in addition to the detector holding device, i.e. the bucky wall stand, with known X-ray devices are patient tables as patient support and positioning devices, which may likewise contain at least one retainer or a slide-in module for an X-ray detector and a manual antiscatter grid in order to enable X-ray images to be taken with a recumbent patient.

### SUMMARY

Against the above-discussed background, an object underlying one or more example embodiments of the present invention is to provide an improved detector holding device and X-ray device by comparison which solves at least some of the cited problems and, with its simple construction and simple handling, offers flexible application options, such as automatic workflows, for example.

To achieve at least this object, a detector holding device and an X-ray device having the features of the independent coordinated claims are provided. Advantageous developments may be derived from the dependent claims.

In a detector holding device of the type cited in the introduction, it is provided, according to one or more example embodiments of the present invention, that the X-ray detector is movable inside the housing in at least one direction in the detector plane via at least one selectable shift actuator.

A housing is proposed which provides the radiolucent wall, in particular panel, and whose interior space is greater than the X-ray detector accommodated therein such that the X-ray detector is displaceable automatically inside the housing along at least one direction via at least one shift actuator. In particular, the housing has a first width and a first height in the detector plane, while the X-ray detector has a smaller second width and/or a smaller second height. In a particularly advantageous embodiment, it is provided in this case that the X-ray detector is displaceable via the at least one shift actuator in a height direction and in a width direction which stand orthogonally to one another.

The ability to move inside the housing is provided in this case in particular via a shift device having the at least one shift actuator. In this case generally known linear actuator or linear actuating mechanism, device and/or means can be used, for example spindle drives, and/or preferably a guided carriage on which the X-ray detector is arranged. During adjustments in two directions perpendicular to one another, in one direction the carriage can for example be moved along the one direction along a guide mechanism, device and/or means (also referred to herein as a guide), for example at least one guide cylinder or tube, while said guide itself, in particular guided on two sides, is linearly adjustable perpendicularly to its course. It is also conceivable to use a carriage which is adjustable in one direction with the aid of a guide, wherein the X-ray detector on the carriage is movable in a direction perpendicular thereto. It is further possible, by rotation of a movement axis established for example by a guide, to provide at least one second direction for translation in the detector plane starting from an orientation in the first direction. Other physical embodiments are of course also conceivable.

In this case the housing is embodied as preferably rectangular, possibly also square, in particular in the detector plane. In a home position in which the detector surface and the radiolucent wall are oriented vertically, the height direction can extend parallel to one side of the rectangle, in particular a longer side, and the width direction perpendicular thereto parallel to the other, in particular shorter, side. In other words, the height direction and the width direction extend at least in a home position along edges of the preferably rectangular housing.

It is therefore proposed to move the X-ray detector practically invisibly inside a housing automatically via at least one shift actuator such that when this at least one degree of freedom, preferably these at least two degrees of freedom, are used there are no moving parts present externally and in particular the detector module itself is not moved. This gives rise to numerous advantages.

For instance, in a panoramic mode of operation (in particular exploiting the ability to control the shift actuator and therefore automatically achievable) in which multiple single images are combined by stitching into one X-ray image, there is no longer any need for a movement on the part of the detector module or of the patient themselves. For example, it can be provided that in a panoramic mode of operation the control device of the X-ray device is designed to select the shift actuator in order to move the X-ray detector into different positions along at least one of the at least one directions and to actuate the X-ray tube assembly and the X-ray detector in order to acquire individual images of a panoramic image at those positions. Given an ability to move in the height direction and in the width direction, such panoramic radiographs are conceivable in both directions. In particular, even with other exposure sequences besides the panoramic mode of operation in the height direction and/or width direction, corresponding to the transverse direction in the home position with a vertically oriented detector surface, a laborious repositioning of the patient is also no longer necessary. One or more example embodiments of the present invention therefore permit a larger or expanded imaging area. Furthermore, new acquisition modes are also conceivable, for example such which permit panoramas with regard to two directions, in particular in a shifting movement along two directions/axes inside the housing.

Moving the X-ray detector inside the housing, i.e. in particular behind the X-ray-permeable wall or panel of the housing, is advantageous in several other respects. For instance, risks due to moving parts are reduced. Additional patient assist devices (“ortho supports”), which provide steps, grab handles and other types of support on account of a moving detector module, are no longer necessary. An improvement in accessibility for persons with limited mobility is achieved. Because more closed, panel-like parts, such as for example the housing, are present, the cleaning of the detector holding device can also be improved.

It should also be noted that X-ray detector holders that are not fixed to a wall of the room in which the X-ray device is installed are also often referred to by the term grid wall unit or “bucky wall stand” or simply “bucky stand”. With this in mind, the detector holding device of one or more example embodiments of the present invention may also be understood as a grid wall unit which provides its functions in their entirety. In any case, however, the described detector holding device is a bucky stand, even when, as described further in the following, the range of application can be expanded as a result of an increase in flexibility.

As is generally known, at least one dose measuring chamber may also be installed inside the housing. As is generally known, the position of the at least one dose measuring chamber can be marked externally on the radiolucent wall.

An antiscatter grid assigned to the X-ray detector and movable at least with the X-ray detector can beneficially be provided in the housing. As is already known, the detector module can therefore also still comprise at least one antiscatter grid which can be moved at least with the X-ray detector. In this case the movement with the X-ray detector is to be understood in the present context in the sense that the antiscatter grid either is or can be motion-coupled with the X-ray detector, in other words is movable by way of the shift actuator, and/or can be moved via dedicated, correspondingly selected actuating elements, in particular, therefore, by at least one grid actuator.

A real, beneficial development in this connection can provide that the antiscatter grid is movable independently of the X-ray detector in the at least one direction via at least one grid actuator. This also enables the position of the antiscatter grid inside the housing to be chosen independently of the position of the X-ray detector, in particular the antiscatter grid to be removed from the beam path of the X-rays to the detector surface when it is not required or, when there are several antiscatter grids, optionally to insert one of them into the beam path. In practice it can be provided for example that the control device of the X-ray device is designed to drive the grid actuator during imaging procedures which are to be performed without an antiscatter grid in order to move the antiscatter grid into a not-in-use position that does not cover the detector surface. In this way a manual handling of the antiscatter grid is no longer necessary, thus providing a significant improvement in terms of convenience in this regard.

With regard to inserting, replacing or removing the antiscatter grid, it can be provided in a beneficial embodiment that the detector module has a presenter arrangement for inserting and removing the antiscatter grid. The presenter arrangement may for example comprise a drawer that is retractable from the housing or some other withdrawal apparatus (i.e. retractable and/or withdrawable apparatus) from which the antiscatter grid can be removed or into which it can be inserted. In particular, the antiscatter grid can be driven via the at least one grid actuator into a presenter position inside the drawer or withdrawal apparatus in order to be presented there. The presenter arrangement can comprise a gripper mechanism, device and/or means (also referred to as a gripper) in order to grip the antiscatter grid for its withdrawal. Just like the grid actuator, the presenter arrangement can be selectable by a control device, for example in response to the actuation of an operator control element of the X-ray device that requests the presentation of the antiscatter grid. The presentation can be performed for example in a similar manner to the presentation of a data medium for a computer, for example a CD.

As already mentioned, the detector holding device is in particular designed such that the detector surface is oriented vertically at least in a home position. In this respect it therefore acts as a bucky wall stand or bucky stand and can be used for example for chest X-rays, for which a bucky wall stand is most frequently used, and the like. However, preferred developments of one or more example embodiments of the present invention provide that the detector holding device offers further, in particular automatically selectable, degrees of freedom which significantly increase the flexibility of application of the detector holding device.

A particularly preferred development of one or more example embodiments of the present invention provides that the stand arrangement comprises a column which carries the detector module and is adjustable in height in the manner of a telescope via a selectable lift actuator. It is therefore proposed to use a telescopic column in which an extremely good utilization of space is given and in particular the space above the detector module remains essentially free such that for example other components of the X-ray device and/or aids can be disposed in an improved arrangement. In particular, as will be discussed in more detail hereinbelow, the use of a telescopic column also permits the detector module to be arranged beneath patient support and positioning devices. In contrast to a conventional column, in which a carriage is guided with the detector module, the possible adjustment path “disappears” inside the column whereby the latter contracts like a telescope when a lower nominal height is desired and consequently does not have to project beyond the detector module.

In this case, where the X-ray detector is movable in the height direction in the housing, the telescopic column can also be dimensioned smaller, i.e. with a lower maximum height, since a part of a nominal height of the X-ray detector can also be provided as a result of the displacement inside the housing. In this context, in order to set a nominal height of the X-ray detector, a control device of the X-ray device can be designed for example to provide a part of the nominal height by way of the shift actuator and a part of the nominal height by way of the lift actuator. In particular, a maximum utilization of the path that can be provided by the shift actuator is conceivable for at least one operating mode, in particular for the acquisition of single images.

In a beneficial development of one or more example embodiments of the present invention, it can further be provided that the detector module is coupled to the column so as to be adjustable in at least one horizontal direction by way of a linear adjustment device having at least one selectable linear actuator. For example, an additional longitudinal displacement can therefore be provided as a further degree of freedom that increases the flexibility.

The detector holding device can preferably be anchorable via an anchorage arrangement in a floor of a room in which the X-ray device is installed. For example, the stand arrangement can therefore have a plinth which in particular carries the telescopic column and for example is anchored in the floor via sufficiently strong bolted connections.

Also basically conceivable, however, is a movability of the detector holding device per se. In such exemplary embodiments it can be provided that the detector holding device has an anchorage arrangement, in particular comprising a rail system, for anchoring the stand arrangement in a floor such that it can be moved in at least one horizontal direction.

In a particularly preferred development of one or more example embodiments of the present invention, it is provided that the detector module is coupled to the stand arrangement so as to be tiltable via a tilt device at least between a or the home position in which the detector surface is oriented vertically and a horizontal position in which the detector surface is oriented horizontally, wherein the tilt device has in particular a selectable tilt actuator effecting the tilting movement. The detector module and consequently the detector surface behind the radiolucent wall can therefore at least also be brought into a horizontal position by tilting by way of corresponding tilting mechanism, device and/or means about a horizontal tilt axis parallel to the detector surface. Instead of the vertical home position, this then produces a horizontal position in which for example a part of the body that is to be imaged can be placed onto the radiolucent wall, for example when conducting hand and/or arm exposures of sitting patients. Particularly advantageously, however, it can be provided, in particular when using a telescopic column of the stand arrangement, that a patient support and positioning device, for example a simple trolley or a simple stretcher, possibly also a patient table, can be driven over the radiolucent wall, in particular over the entire detector module, such that the detector holding device can also be used for example for acquisitions with a recumbent patient and a further X-ray detector in a patient table is possibly no longer required. In other words, in this way the detector holding device can provide a kind of two-in-one solution since it not only functions as a bucky wall stand but can also be used with recumbent patients in combination with a patient support and positioning device. For example, instead of a sophisticated patient table, which could for example have a slide-in module for an X-ray detector or an X-ray detector itself, this enables a significantly simpler patient support and positioning device, for example a simple trolley, to be used. Preferably, with a view to the possible automation of workflows, the tilt device may comprise a tilt actuator that is selectable by the control device of the X-ray device.

It should be noted that in exemplary embodiments, in addition to the home position and the horizontal position, further tilting positions can also be set via the tilt device or in actual fact by the tilt actuator, for example a tilting position by −20° for specialized X-ray image acquisitions and the like.

In order to provide a further degree of freedom further increasing the flexibility of application, it can be provided that the detector module is coupled to the stand arrangement by way of a swivel device which permits the detector module to be swiveled about a swivel axis that is perpendicular to the detector surface, the swivel device having in particular a selectable swivel actuator effecting the swiveling movement. This is in particular beneficial when the housing has a rectangular shape parallel to the detector plane in which one dimension, in particular the first height, is greater, in particular significantly greater, than a second dimension, in particular the first width. This means in fact that in the home position a significantly more extensive movability of the X-ray detector in the height direction is possible than in the width direction (if indeed provided). If, however, single images succeed one another in the width direction, for example in a panoramic radiograph, and if the width direction were not sufficient to cover all the exposure positions in this direction, a swiveling movement about the swivel axis perpendicular to the detector surface, in particular by 90°, can lead to this still being made possible. A repositioning of the patient is then not necessary. In particular, therefore, there exists in addition to the home position at least one further swivel position in which the X-ray detector is swiveled through 90°, wherein swivel positions lying therebetween or going beyond them can also be conceivable. In particular, in the further swivel position, the longer edge of the housing can extend horizontally, whereas in the home position it extends vertically. A swivel actuator selectable by the control device is preferably provided here also in order to automate and simplify workflows.

A swivel device is in particular advantageous also when a use of the detector holding device is provided for lateral exposures with recumbent patients, in which the X-ray detector standing at least substantially vertically is arranged at the side next to the patient or the corresponding patient support and positioning device. In contrast to the standing patient, the patient's longitudinal axis then extends horizontally such that in order to acquire a series of images along the longitudinal axis of the patient, the long edge of the housing is beneficially also oriented horizontally and consequently the longest possible adjustment path in the at least one direction, in this case in particular the height direction of the detector module, is provided. This is possible via the swivel device.

In this regard, when both the swivel device and the tilt device as well as the linear adjustment device or when two of these devices are provided, it is ultimately irrelevant in which order these are installed between the detector module and the stand arrangement, in particular the column. Preferably, starting from the detector module, firstly the swivel capability is realized, then the tilt capability, then, if provided, the linear adjustability. It goes without saying that, in particular given also possible manual adjustability in each case of the swivel device, the tilt device and the linear adjustment device, respective lock or locking mechanism, device and/or means for locking the detector module in the respective position can be assigned.

In a beneficial development it can be provided that the detector module, in particular the housing, has at least one grab handle for an examination subject, i.e. a patient. Such grab handles, which are also already known from known prior art bucky wall stands, can also be beneficially provided within the scope of the present invention in order to assist in assuming and/or maintaining a position during the acquisition of X-ray images. In particular, on account of the movement of the X-ray detector inside the housing, these grab handles or the housing per se can also be used further without restriction when acquisition procedures with a moving X-ray detector are pending.

In this connection it should further be pointed out that the detector holding device, according to one or more example embodiments of the present invention, as a bucky wall stand or as a substitute for a bucky wall stand, is preferably not designed to support the weight of an adult completely, as would be the case for example with a patient table. For example, it can be provided that the detector holding device can take a maximum load of less than 60 kg, in particular less than 50 kg, as is usual for standing patients with grab handles.

In actual example embodiments it can be provided that the housing has a dimension of 50 to 100 by 50 to 150 cm in the detector plane and/or that the X-ray detector has a dimension of 20 to 50 by 20 to 80 cm in the detector plane. For example, a housing measuring 60 by 120 cm in size, in particular width by height, is conceivable in which a detector having a detector surface measuring 25 by 50 cm in size is movable. In another example, the housing can be 81 by 144 cm (in particular width by height) in size, while the X-ray detector or the detector surface can measure 30 by 53 cm.

It is particularly advantageous if all the actuators used to implement the degrees of freedom are selectable in order, as indicated already to some degree, to enable automated workflows to be realized as far as possible via the control device of the X-ray device.

An X-ray device, according to one or more example embodiments of the present invention, comprises a tube assembly holding device having at least one X-ray tube assembly and at least one selectable tube assembly actuator for positioning the X-ray tube assembly, a detector holding device according to one or more example embodiments of the present invention, and a control device. All statements made with regard to the detector holding device can be applied analogously to the X-ray device and vice versa.

In this regard, the control device is preferably able to drive the actuating elements of the detector holding device in such a way that the position of the X-ray detector, in particular of the detector surface, follows the position of the X-ray tube assembly (obviously including also its orientation). In this way the X-ray detector is positioned correctly relative to the X-ray tube assembly at all times, thereby significantly reducing the risk of inaccurate acquisition operations. In particular it can be provided that the control device of the X-ray device is configured to initiate a coordinated movement of the X-ray tube assembly and the X-ray detector for receiving X-ray radiation from the X-ray tube assembly via the X-ray detector. In this way it is in particular also possible to realize, as it were, “exposure trajectories” from exposure position to exposure position in unison, for example for panoramic images or the like, and to achieve an extremely comprehensive degree of automation.

In a particularly beneficial development of one or more example embodiments of the present invention, the X-ray device additionally has at least one patient table and/or at least one mobile patient support and positioning device. In particular, as already stated above, the detector holding device in this arrangement is designed such that, for example through the provision of the tilt capability, the X-ray detector of the detector holding device can also be used for acquiring images in which the patient is lying on the mobile patient support and positioning device or the patient table. It is particularly advantageously provided in this case that the detector holding device is dimensioned such that in at least one setting with the detector module in the horizontal position a lying surface of the patient table and/or of the mobile patient support and positioning device can be placed over the radiolucent wall of the housing. Particularly beneficially, the stand arrangement then has the described telescopic column such that the detector holding device as it were contracts as small as possible and no or at least no relevant portions of the stand arrangement protrude beyond the detector module such that the entire area above the detector module is available for the placement of the lying surface of the patient table and/or the mobile patient support and positioning device, in particular a trolley or stretcher. The detector holding device then forms a sort of two-in-one solution which provides the X-ray detector both for acquiring images with the patient lying on the patient support and positioning device and also for acquiring images with a standing patient in its function as a bucky stand.

In a beneficial development of one or more example embodiments of the present invention, it can further be provided that the X-ray device has at least one camera, the control device being designed to control at least one actuator of the X-ray detector holding device as a function of image data of the camera. Owing to the versatile, selectable degrees of freedom of the detector holding device it is therefore possible also to realize camera-based automated workflows which, for example in a change in position of the patient by controlling the actuating elements, in particular of both the tube assembly holding device and the detector holding device, the exposure arrangement can follow in such a way that the image acquisition can be continued.

### BRIEF DESCRIPTION OF THE DRAWINGS

Further advantages and specific details of the present invention will become apparent from the exemplary embodiments described in the following, as well as with reference to the drawings, in which:

FIG. 1 shows a schematic front view of a detector holding device, according to one or more example embodiments of the present invention, with the detector module in the home position,

FIG. 2 shows a schematic side view of a detector holding device, according to one or more example embodiments of the present invention, with the detector module in the home position,

FIG. 3 shows a schematic cross-sectional view through the detector module,

FIG. 4 shows partial images depicting the detector module of the detector holding device in different swivel positions,

FIG. 5 shows a view of a first exemplary embodiment of an X-ray device, according to one or more example embodiments of the present invention, in a first operating state,

FIG. 6 shows a view of the first exemplary embodiment in a second operating state,

FIG. 7 shows a view of a second exemplary embodiment of an X-ray device, according to one or more example embodiments of the present invention, in a first operating state, and

FIG. 8 shows a view of the second exemplary embodiment in a second operating state.

### DETAILED DESCRIPTION

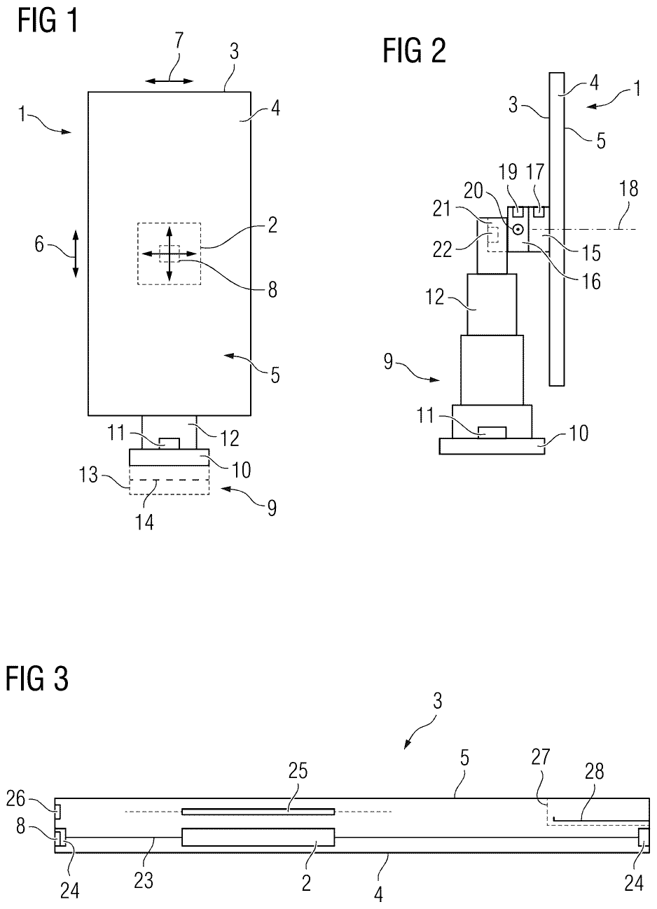

FIG. 1 and FIG. 2 show schematic diagrams of a detector holding device 1, according to one or more example embodiments of the present invention, in a front view and a side view, wherein a detector module 3 containing the X-ray detector 2 indicated by a dashed outline in FIG. 1, the housing 4 of said detector module 3 enclosing the X-ray detector 2, is located in a home position. In this home position, the radiolucent front plate-like wall 5 (panel) which extends over the detector surface of the X-ray detector 2 is oriented vertically. This means a detector plane in which the detector surface lies likewise extends vertically. The housing 4 and therefore also the radiolucent wall 5 have a rectangular shape in the detector plane, wherein the housing 4 has a first height in a height direction 6 of the detector module 3 and a first width in a width direction 7 of the detector module 3. As can be seen, the first height, i.e. the dimension in the height direction 6, is greater than the first width, i.e. the dimension in the width direction 7. The X-ray detector 2 has a second width and a second height which are clearly smaller in each case than the first width and the first height. The X-ray detector 2 is movable inside the housing 4 both in the height direction 6 and in the width direction 7 (in the home position, a transverse direction) by way of at least one corresponding selectable shift actuator 8, in this case indicated only by a dashed outline.

The detector module 3 is carried by a stand arrangement 9, which in the present example has a plinth 10 which can be anchored to or in a floor, as well as a column 12 that is adjustable in height in the manner of a telescope (hereinbelow: telescopic column 12) via a selectable lift actuator 11. The anchoring arrangement 13 provided for the anchoring can comprise for example a bolted connection for static anchoring, though optionally it may also permit a movability in a horizontal direction in the floor, for example via a rail system 14.

As can be seen from FIG. 2, the detector module 3 is secured to the telescopic column 12 by way of a swivel device 15 and a tilt device 16. The swivel device 15 permits, where necessary manually and in the present example in any case via a selectable swivel actuator 17, the detector module 3 to be swiveled about a swivel axis perpendicular to the detector surface, also indicated in FIG. 2, in particular starting from the home position into a further swivel position in which the detector module 3 is swiveled through 90° such that the long edge of the housing 4, i.e. the height direction 6 of the detector module 3, now extends horizontally and the shorter edge of the housing 4, i.e. the width direction 7 of the detector module 3, now extends vertically.

The tilt device 16 accordingly has a selectable tilt actuator 19 in order to tilt the detector module 3 out of the vertical home position, in the present example about a horizontal tilt axis 20 lying parallel to the detector surface. If necessary, a manual tilting movement can additionally be realized here also. A tilting is possible at least into a horizontal position in which the detector surface and the radiolucent wall 5 extend horizontally, in which case the radiolucent wall 5 then forms the top side. Then, both the width direction 7 of the detector module 3 and the height direction 6 of the detector module 3 therefore extend horizontally. A tilting into intermediate positions or into the other tilt direction is also conceivable for specialized X-ray image acquisitions.

Optionally, FIG. 2 also shows a linear adjustment device 21 having at least one selectable linear actuator 22 for linear adjustment of the detector module 3 in at least one horizontal direction, in particular in a longitudinal direction perpendicular to the width direction 7 and the height direction 6 in the home position. A telescopic arm can be used, for example.

FIG. 3 shows the detector module 3 in more detail in a schematic cross-sectional view. As can be seen, the X-ray detector 2 is arranged in its interior as movable via a shift device. In this arrangement, generally known linear actuator or linear actuating mechanism, device and/or means can be used, for example spindle drives and/or preferably a guided carriage on which the X-ray detector 2 is arranged. In the present example, the carriage can be moved along a guide 23 (also referred to as a guide mechanism, device and/or means) in one direction, in particular i.e. the width direction 7 or the height direction 6, whereas said guide 23 itself is linearly adjustable, in particular on both sides, perpendicularly to its course by way of linear actuator 24. In other exemplary embodiments it is also conceivable that a carriage is guided in or by the linear actuator 24 on which the X-ray detector 2 can be moved in a direction perpendicular hereto by way of the guide 23. The shift actuator 8 can be implemented via motors, for example.

Inside the housing 4, movable in another plane parallel to the detector plane, the X-ray detector 2 is assigned an antiscatter grid 25 which in the present example is shown in a position upstream of the X-ray detector 2 and can be moved jointly with the latter. In the present case, however, it is also possible, for example by way of a comparable further shift device, for the antiscatter grid 25 to be movable independently in the height direction 6 and the width direction 7, wherein in the present instance only the corresponding at least one selectable grid actuator 26 is indicated. By this means it is possible to move the antiscatter grid 25 into a position that does not overlap with the detector surface when it is not required. In the present example, a presenter arrangement 27 is also provided for removing, adding or replacing the antiscatter grid 25. The presenter arrangement 27 has a drawer 28 (or other withdrawal apparatus) which is retractable from the housing 4, from which the antiscatter grid 25 can be removed or into which it can be inserted. The antiscatter grid 25 can be moved via the at least one grid actuator 26 into a presenter position inside the drawer 28 or withdrawal device in order to be presented there. The presenter arrangement 27 may comprise a gripper mechanism, device and/or means (not shown) in order to grip the antiscatter grid 25 for its withdrawal.

It should be noted that at least one dose measuring chamber, not shown in more detail here for clarity of illustration reasons, may also be arranged in the housing 4.

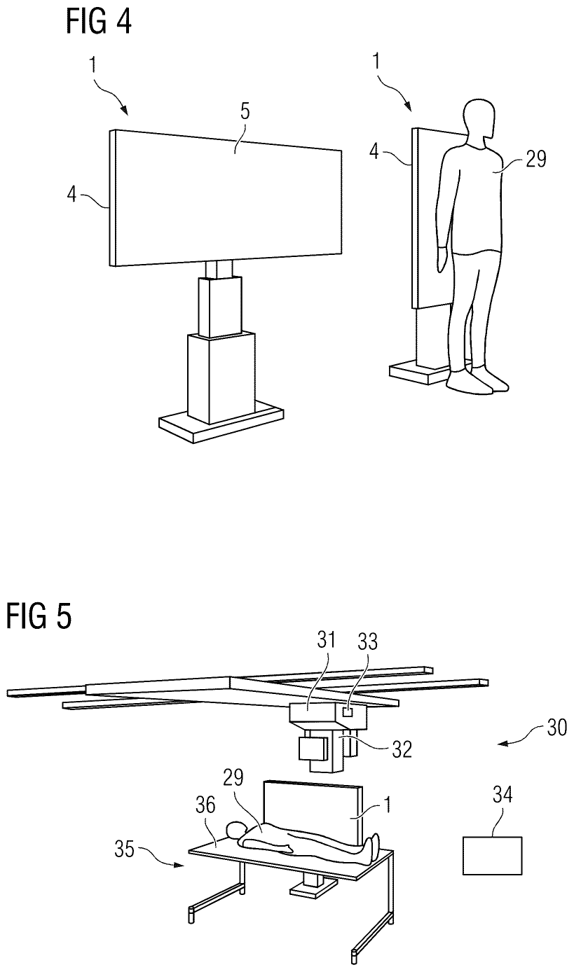

To provide a more detailed explanation, FIG. 4 shows the X-ray detector holder 1 in two different swivel positions. In this case the image on the right shows the detector holding device 1 in the home position with a patient 29 standing in front of the detector holding device 1, as is known with bucky wall stands. To provide better support for the patient when assuming and/or holding a position for the image acquisition, at least one holding mechanism, device and/or means (not shown for clarity of illustration reasons) may also be provided on the housing 4.

The image on the left in FIG. 4 shows the detector holding device 1 in the further swivel position already discussed above, i.e. rotated by 90° about the swivel axis 18. The further swivel position was assumed by driving the swivel actuator 17 in an appropriate manner. It is suitable in particular, as will be explained in more detail later, for lateral X-ray image acquisitions with a recumbent patient 29.

FIG. 5 shows a first exemplary embodiment of an X-ray device 30, according to one or more example embodiments of the present invention. The X-ray device 30 comprises a tube assembly holding device 31 which has an X-ray tube assembly 32 with collimator and in the present example is mounted on a ceiling of the room in which the X-ray device 30 is installed. The X-ray tube assembly 32 with collimator can be moved into different positions by way of at least one selectable tube assembly actuator 33. In addition to the tube assembly holding device 31, the X-ray device 30 also comprises the detector holding device 1 described with reference to FIGS. 1 to 4 as well as a control device 34, as is indicated only schematically in the present example. The actuating elements of the tube assembly holding device 31 and the detector holding device 1, in particular, therefore, the at least one shift actuator 8, the swivel actuator 17, the tilt actuator 19, the linear actuator 22, the lift actuator 11 and the grid actuator 26, can be controlled via the control device 34. With regard to the X-ray detector 2, six degrees of freedom can be controlled: the shift capability in the height direction 6, the shift capability in the width direction 7, the swivel capability about the swivel axis 18, the tilt capability about the tilt axis 20, the longitudinal displacement with the linear actuator 22, and the height adjustment via the lift actuator 11. In addition, the control device 34 can control the X-ray tube assembly 32 and the X-ray detector 2 for the acquisition of X-ray images. By driving the actuating elements and controlling the image acquisition mode it is possible to automate numerous workflows.

In the present example, the X-ray device 30 also comprises, as a mobile patient support and positioning mechanism, device and/or means 35, a trolley 36 of simple design on which a patient 29 can be supported in a recumbent position. In the view shown in FIG. 5, the detector module 3 is located in the further swivel position and is arranged at the side of the mobile patient support and positioning device 35 so that lateral X-ray images of the patient 29 can be produced. Owing to the use of the further swivel position, these images can be acquired in many positions along the longitudinal direction of the patient 29 since the height direction 6, in which a greater movement range is available, corresponds to this longitudinal direction. For example, a panoramic radiograph can be produced by the acquisition of multiple individual images in the longitudinal direction of the patient, wherein the control device 34 can control the movement of the X-ray detector 2 and the X-ray tube assembly 32 just like its acquisition mode of operation. In this process the X-ray tube assembly 32 is correctly tracked by the X-ray detector 2 at all times such that the X-rays emitted by the X-ray tube assembly 32 can be received by the X-ray detector 2 on its detector surface. In particular the coordinate systems of the holding devices 1, 31 are registered with one another for this purpose.

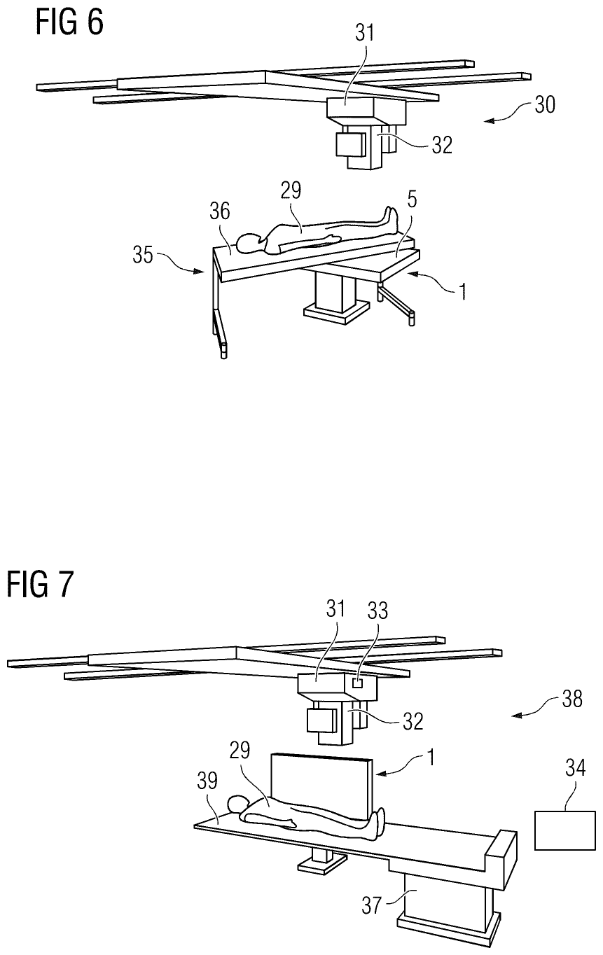

FIG. 6 shows the X-ray device 30 in a further operating state. In this case the detector module 3 is tilted into the horizontal position. Owing to the telescopic column 12, the stand arrangement 9 does not project beyond the detector module 3 and the patient support and positioning surface of the mobile patient support and positioning device 35 can be positioned without difficulty above the radiolucent wall 5 in order to acquire X-ray images with the patient 29 lying in the anterior-posterior direction.

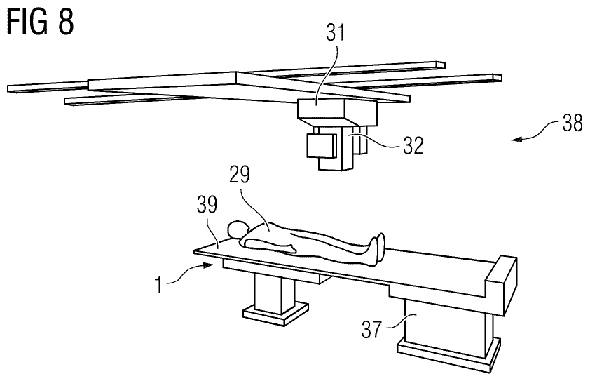

FIGS. 7 and 8 show a second exemplary embodiment of an X-ray device 38 in corresponding operating states, wherein, in contrast to the X-ray device 30, instead of the mobile patient support and positioning device 35, in the case of the X-ray device 38 a permanently installed patient table 37 is used, though the latter itself has no X-ray detector 2 or slide-in module for this purpose. As can be seen, in such a case according to FIG. 7, the detector holding device 1 can also be used for lateral X-ray image acquisitions, and according to FIG. 8, when positioned below the tabletop 39, for anterior-posterior X-ray image acquisitions on a recumbent patient 29.

Both in the case of the X-ray device 30 and in the case of the X-ray device 38, the detector holding device 1 can of course also be used for acquiring X-ray images with a standing patient 29, as illustrated on the right in FIG. 4.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternatively, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” or “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A detector holding device for an X-ray device, the detector holding device comprising:
an X-ray detector; and
a stand arrangement by which the X-ray detector is carried inside a housing of a detector module, wherein
the X-ray detector has a detector surface extending in a detector plane and being arranged behind a radiolucent wall of the housing, and
the X-ray detector is movable inside the housing in at least one direction in the detector plane via at least one selectable shift actuator.

2. The detector holding device as claimed in claim 1, wherein the X-ray detector is configured to be shifted, via the at least one selectable shift actuator, in a height direction and in a width direction, the height direction being perpendicular to the width direction.

3. The detector holding device as claimed in claim 1, further comprising:
an antiscatter grid in the housing, the antiscatter grid assigned to the X-ray detector and movable at least with the X-ray detector.

4. The detector holding device as claimed in claim 3, wherein the antiscatter grid is movable independently of the X-ray detector in the at least one direction via at least one grid actuator.

5. The detector holding device as claimed in claim 4, wherein the detector module has a presenter arrangement for inserting and removing the antiscatter grid.

6. The detector holding device as claimed in claim 1, wherein the detector surface is oriented vertically at least in a home position.

7. The detector holding device as claimed in claim 1, wherein the stand arrangement comprises:
a column configured to carry the detector module and being adjustable in height in a manner of a telescope via a selectable lift actuator.

8. The detector holding device as claimed in claim 7, wherein the detector module is coupled to the column so as to be adjustable in at least one horizontal direction by way of a linear adjustment device having at least one selectable linear actuator.

9. The detector holding device as claimed in claim 1, further comprising:
an anchoring arrangement configured to anchor the stand arrangement in a floor so as to be movable in at least one horizontal direction.

10. The detector holding device as claimed in claim 1, wherein the detector module is coupled to the stand arrangement via a tilt device so as to be tiltable at least between a home position in which the detector surface is oriented vertically and a horizontal position in which the detector surface is oriented horizontally, and wherein the tilt device has a selectable tilt actuator effecting a tilting movement.

11. The detector holding device as claimed in claim 1, wherein
the detector module is coupled to the stand arrangement by a swivel device,
the swivel device permits the detector module to be swiveled about a swivel axis that is perpendicular to the detector surface, and
the swivel device has a selectable swivel actuator effecting a swiveling movement.

12. The detector holding device as claimed in claim 1, wherein the detector module has at least one grab handle for a patient.

13. The detector holding device as claimed in claim 1, wherein at least one of
the housing has a dimension of 50 to 100 by 50 to 150 cm in the detector plane, or
the X-ray detector has a dimension of 20 to 50 by 20 to 80 cm in the detector plane.

14. An X-ray device comprising:
a tube assembly holding device having at least one X-ray tube assembly and at least one selectable tube assembly actuator to position the at least one X-ray tube assembly;
the detector holding device as claimed in claim 1; and
a control device.

15. The X-ray device as claimed in claim 14, further comprising at least one of:
at least one patient table, or
at least one mobile patient support and positioning device.

16. The detector holding device as claimed in claim 9, wherein the anchoring arrangement is a rail system.

17. The detector holding device as claimed in claim 12, wherein the at least one grab handle is located on the housing.

18. The detector holding device as claimed in claim 5, wherein the stand arrangement comprises:
a column configured to carry the detector module and being adjustable in height in a manner of a telescope via a selectable lift actuator.

19. The detector holding device as claimed in claim 5, wherein the detector module is coupled to the stand arrangement via a tilt device so as to be tiltable at least between a home position in which the detector surface is oriented vertically and a horizontal position in which the detector surface is oriented horizontally, and wherein the tilt device has a selectable tilt actuator effecting a tilting movement.

20. The detector holding device as claimed in claim 5, wherein
the detector module is coupled to the stand arrangement by a swivel device,
the swivel device permits the detector module to be swiveled about a swivel axis that is perpendicular to the detector surface, and
the swivel device has a selectable swivel actuator effecting a swiveling movement.
